(19) United States
(12) Patent Application Publication
(10) Pub. No.: US 2021/0158909 A1
(43) Pub. Date: May 27, 2021

(54) PRECISION COHORT ANALYTICS FOR PUBLIC HEALTH MANAGEMENT

(71) Applicant: International Business Machines Corporation, Armonk, NY (US)

(72) Inventors: Kenney Ng, Arlington, MA (US); Paul C. Tang, Los Altos, CA (US); Sarah Miller, Cambridge, MA (US)

(21) Appl. No.: 16/693,540

(22) Filed: Nov. 25, 2019

(51) Int. Cl. G16H 10/60; G16H 50/20

(52) U.S. Cl. CPC G16H 10/60; G16H 50/20

(57) ABSTRACT

Provided is a method, a computer program product, and a system for generating personalized treatment options and associated outcome estimates for patients. The method includes retrieving information from an electronic health records database relating to a patient cohort, detecting decision points from the information retrieved, and computing actual treatment options from the decision points. The method also includes computing precision cohort treatment options from the decision points using precision cohort analytics on a patient group and analyzing the decision points by comparing the actual treatment options with the precision cohort treatment options for each of the decision points to determine recommended measures. The method further includes generating a precision population analysis report based on the recommended measure.

### BACKGROUND

The present disclosure relates to population health management, and more specifically, to generating personalized treatment options and associated outcome estimates for patients.

Population health management relates to the application of proactive interventions for defined cohorts of patients in an attempt to maintain or improve their health. An organization can utilize analytics and insights to better manage and improve population health while lowering costs. A patient cohort refers to any group of individuals affected by common diseases, environmental or temporal influences, treatments, or other traits whose progress can be assessed.

### SUMMARY

Embodiments of the present disclosure include a computer implemented method for generating personalized treatment options and associated outcome estimates involving patients. The computer implemented method includes retrieving information from an electronic health record database relating to a patient cohort, identifying decision points from the information retrieved, and computing actual treatment options from the decision points. The computer implemented method also includes computing precision cohort treatment options from the decision points using precision cohort analytics on a patient group and analyzing the decision points by comparing the actual treatment options with the precision cohort treatment options for each of the decision points to determine recommended measures. The patient group can be chosen from a group consisting of a population cohort, a separate patient cohort, a patient panel, and from an individual. The computer implemented method further includes generating a precision population analysis report based, at least partially, on the recommended measures.

Further embodiments are directed to a computer program product for generating personalized treatment options and associated outcome estimates involving patients, which can include a computer readable storage medium having program instruction embodied therewith, the program instruction executable by a processor to cause the processor to perform a method. The method includes retrieving information from an electronic health record database relating to a patient cohort, identifying decision points from the information retrieved, and computing actual treatment options from the decision points. The method also includes computing precision cohort treatment options from the decision points using precision cohort analytics on a patient group and analyzing the decision points by comparing the actual treatment options with the precision cohort treatment options for each of the decision points to determine recommended measures. The patient group can be chosen from a group consisting of a population cohort, a separate patient cohort, a patient panel, and from an individual. The method further includes generating a precision population analysis report based, at least partially, on the recommended measures.

Additional embodiments are directed to a precision cohort analytics system for generating personalized treatment options and associated outcome estimates involving patients, including at least one processor and at least one memory component. The system also includes a records retriever configured to retrieve information from an electronics health record database relating to a patient cohort and a treatment calculator configured to compute actual treatment options from decision points identified in the information on a patient group. The system also includes a precision cohort calculator configured to compute precision cohort treatment options from the decision points and a treatment analyzer configured to analyze the decision points by comparing the actual treatment option with the precision cohort treatment options to determine recommended measure for the patient cohort. The system further includes a report generator configured to generate a precision population analysis report based on the recommended measures determined.

### BRIEF DESCRIPTION OF THE DRAWINGS

These and other features, aspects, and advantages of the embodiments of the disclosure will become better understood with regard to the following description, appended claims, and accompanying drawings where:

FIG. 1 is a block diagram illustrating an exemplary longitudinal data timeline for a patient, in accordance with embodiments of the present disclosure.

FIG. 2 is a block diagram illustrating a precision cohort analytics system, in accordance with embodiments of the present disclosure.

FIG. 3 is an exemplary precision population analysis report illustrating summary statistics and metrics, in accordance with embodiments of the present disclosure.

FIG. 4 is an exemplary precision population analysis report illustrating clinical inertia, in accordance with embodiments of the present disclosure.

FIG. 5 is an exemplary precision population analysis report illustrating impact and value, in accordance with embodiments of the present disclosure.

FIG. 6 is a flow chart of a precision cohort analysis process, in accordance with embodiments of the present disclosure.

FIG. 7 is a high-level block diagram illustrating an example computer system that may be used in implementing one or more of the methods, tools, and modules, and any related functions, described herein, in accordance with embodiments of the present disclosure.

FIG. 8 depicts a cloud computing environment, in accordance with embodiments of the present disclosure.

FIG. 9 depicts abstraction model layers, in accordance with embodiments of the present disclosure.

While the present disclosure is amenable to various modifications and alternative forms, specifics thereof have been shown by way of example in the drawings and will be described in detail. It should be understood, however, that the intention is not to limit the particular embodiments described. On the contrary, the intention is to cover all modifications, equivalents, and alternatives falling within the scope of the present disclosure. Like reference numerals are used to designate like parts in the accompanying drawings.

### DETAILED DESCRIPTION

The present disclosure relates to population health management, and more specifically, to generating personalized treatment options and associated outcome estimates for patients. While the present disclosure is not necessarily limited to such applications, various aspects of the disclosure may be appreciated through a discussion of various examples using this context.

Population health management is a term used in the healthcare industry used to describe a discipline that studies and facilitates care delivery across a population of individuals. A goal of population health management is to gather, normalize, and analyze the health records and clinical data of patients to uncover possible opportunities to improve the health of patients and to reduce financial burdens of healthcare providers. Evidence-based decisions are made by aggregating, exchanging, and analyzing patient data to coordinate care and promote the wellness of patients.

Data analytics, including predictive analytics, are used in population health management to determine actionable steps for providers and operational data needs across an organization. These analytics can provide insights, allowing providers to identify and address healthcare deficiencies within a patient population.

To improve financial burdens for healthcare providers, population health management attempts to mitigate costs by focusing on an appropriate utilization of services to manage and coordinate care. For instance, chronic diseases within a population can be mitigated by effectively managing and possibly preventing those chronic diseases.

Healthcare deficiencies, or care gaps, can be identified using the data analytics, including process metrics. These metrics include, and without limitation, delivery of care services metrics and outcome metrics. By evaluating the analytics and metrics, healthcare providers can identify needs for a given patient population. For example, a majority of a patient population may have heart disease and chronic liver disease. Once identified, the healthcare provider can devise a plan to help mitigate those illnesses and lower overall costs.

One possible way that population health management assesses patients is through risk stratification. Risk stratification involves dividing a population of patients into groups based on their vital health indicators, lifestyle, and medical history. Several different methods can be used when implementing risk stratification. These methods of risk stratification include, but are not limited to, hierarchical condition categories (HCCs), adjusted clinical groups (ACG), elder risk assessment (ERA), chronic comorbidity count (CCC), and Charlson comorbidity measure. Typically, risk stratification methods are based, at least partially, on comorbidity. Comorbidity refers to the simultaneous presence of two or more chronic diseases or conditions in a patient.

Limitations on precision remain, however, in population health management techniques. For example, current population health management approaches and insights are not precise or actionable regarding specific patient cohorts. Most approaches merely attempt to identify patients who might incur large healthcare expenses at some future point. These identified patients are managed by notifying them to encourage some generic action.

Embodiments of the present disclosure may overcome the above, and other problems, by using a precision cohort analytics system. The precision cohort analytics system is configured to process patient level longitudinal data from electronic health records using precision cohort analytics to generate personalized treatment options. The precision cohort analytics system is further configured to generate associated outcome estimates using the precision cohort analytics. The associated outcome estimates can be compared to the actual treatments to identify insights that can better describe and quantify population health management issues. These issues include clinical inertia, missed opportunities for improved outcomes based on treatment option changes, and potential impacts, in terms of time and costs, of the missed opportunities.

More specifically, the precision cohort analytics system described herein applies precision cohort analytics to a patient group to determine actionable healthcare deficiencies within a patient cohort. A patient cohort, or population cohort, as described herein refers to a group of individuals affected by common diseases, environmental or temporal influences, treatments, or other traits whose progress can be assessed. In other words, the precision cohort analytics system searches electronic health records for a given patient cohort to detect effective treatment options. Accordingly, actual treatment options given to the patient cohort are compared to precision cohort treatment options to determine where changes can be made. Additionally, insights can be gained into clinical inertia, missed opportunities for improved outcomes, and potential impacts of those missed opportunities.

By way of example, a patient cohort is defined by healthcare provider, as having diabetes and liver disease. The patient cohort receives certain treatments for their illnesses depending on the decisions made by their doctors. To analyze the treatments, the precision cohort analytics system retrieves health records relating to the patient cohort. The health records can be analyzed to identify decision points in those health records that doctors made regarding the patients' illness. Based on the decision points, actual treatment options taken by the patients can be computed. This allows for an analysis of how effective the current medical treatment options being given are for the patients. Also based on the decision points, precision cohort treatment options can be computed. Using precision cohort analytics, a more precise definition of a cohort can refine the treatments given to a specific grouping of patients. By analyzing those treatments and the effectiveness of those treatments, an alternative treatment option can be devised for the patient cohort. A comparison can be made between the actual treatment options and the precision cohort treatment options to determine recommended measures that can be taken.

Additionally, or alternatively, further analysis and reporting can be generated by the precision cohort analytics system. An analysis of the decision points can be made to determine the prevalence of clinical inertia. Clinical inertia refers to the failure to establish appropriate targets and escalate treatment to achieve treatment goals. Also, the treatment options can further be compared to quantify the amount of treatment decision overlap that has occurred. The outcomes can also be further compared to quantify differences in expected control of the conditions.

In some embodiments, the precision cohort treatment options can be performed for different patient groups. The different patient groups include, and without limitation, population groups, different patient cohorts, patient panels, as well as individual patients.

In some embodiments, retrieving information from an electronic health record database includes identifying clinical treatment decision points within the information. The clinical treatment decision points can be listed as events of longitudinal histories relating to patients. A longitudinal history, as referred herein describes a clinical summary of a patient over a period of time. Characteristic features for each patient included in the patient cohort can be retrieved that relate to the clinical treatment decision points. For example, a decision point may be a treatment method for diabetes. The characteristic features for a patient can include the type of medication, the dosage, the physical characteristics of the patient such as weight, age, and height. The outcome features associated with outcomes relating to the clinical treatment decision points can also be retrieved. For example, referring back to the previous example of a treatment method for a diabetes decision point. The outcome of that treatment is that the diabetes remains uncontrolled. The medical condition of the patient, the treatment options being given, as well as other types of characteristics can be considered characteristic features which can also be obtained and used to perform precision cohort analytics.

In some embodiments, computing actual treatment options includes identifying actual treatment options taken for each of the decision points identified in the retrieved information taken from an electronic health record database. For example, a decision point may relate to a patient with prostate cancer. Treatment options can include radiation, hormone therapy, surgery, chemotherapy, and the like. The treatment option taken can be the treatment the doctor recommended the patient follow for their prostate cancer. Once the treatment options taken have been determined, the associated outcomes for those treatment options can be computed. Referring back to the previous example, the associated outcomes relating to the patients with prostate cancer can be whether the patient is responding to the treatment options or whether the illness remains uncontrolled.

In some embodiments, computing precision cohort treatment options includes applying precision cohort analytics to each of the decision points identified in the retrieved information taken from an electronic health record database. A precision cohort groups patients based on a quantitative measure of similarity between patients in terms of a particular clinical outcome. The precision cohort analytics can generate personalized treatment options based on the decision points. Once generated, treatment option suggestion can be chosen from the generated personalized treatment options. These suggestions can be the treatment options which have the highest likelihood of a positive outcome. The associated outcomes for the treatment option suggestions can be estimated as well. The associated outcomes can be estimated on causal inference methods. Causal inference refers to a process of drawing a conclusion about a causal connection based on the conditions of the occurrence of an effect.

In some embodiments, analyzing the decision points includes determining a clinical inertia by analyzing the decision points with no treatment change. The clinical inertia can be an indicator that treatment has failed to establish appropriate targets or escalate treatments to achieve a treatment goal. If no treatment change is identified, the outcome can be observed for a decision to determine if clinical inertia applies. Additionally, a comparison can be made of the actual treatment options with precision cohort treatment options to quantify an amount of treatment decision overlap. The comparison can assist healthcare providers in determining if the actual treatment options are the correct course of action for a given patient cohort. The outcomes can also be compared to further give insight into how closely the results of the actual treatment options are with the precision treatment options. A potential impact on executing the precision treatment options over the actual treatment options can be estimated based on the cost and efficiency of the treatment options.

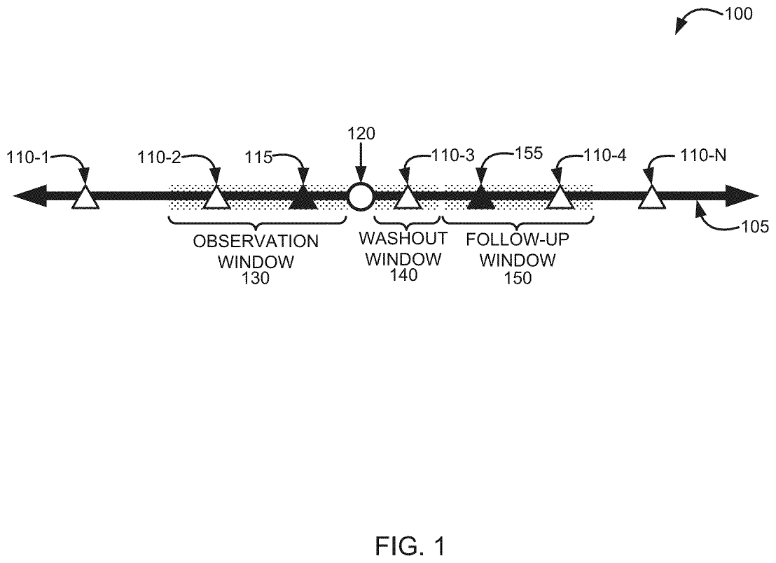

Referring now to FIG. 1, shown is a diagrammatic illustration of a longitudinal history 100 for a patient, in accordance with embodiments of the present disclosure. The longitudinal history 100 includes a timeline 105, events 110-1, 110-2, 110-3, 110-4, 110-N (collectively “events 110”), where N is a variable integer representing any number of possible events 110, uncontrolled event 115, a decision point 120, an observation window 130, a washout window 140, a follow-up window 150, and an outcome event 155.

The timeline 105 is an illustrative representation of time for a which a patient has received treatment. The timeline 105 is oriented around an event-based timeline anchored to a key event. Events 110-1, 110-2, 110-3, 110-4, 110-N are events along the longitudinal history which represent some kind of medical treatment provided to a patient. For example, event 110-1 may be a doctor visit for a physical, event 110-2 may be a visit to a dermatologist, and so on. The events 110 refer to medical events along the timeline 105 of the longitudinal history 100 which may not relate to the decision point 120. Individual patient data are documented as events 110 from various sources and are arranged in calendar time. In some embodiments, the events 110 are encounters with a healthcare system that are captured by an electronic health record system and can be sorted by service date in calendar time. The events 110 can be associated with information on medical services, diagnoses, procedures, and similar events. Payments or charges can also be included within the information for the events 110.

The uncontrolled event 115 is an illustrative representation of an illness, symptom, or disease that is not under control. An uncontrolled illness refers to an illness that is not responding to current treatment options given to a patient. The uncontrolled event 115 can include any illness symptom, condition, or disease which is relevant to the decisions made at the decision point 120.

The decision point 120 represents a response by a medical professional to the uncontrolled event 115. In some embodiments, data related to the decision point 120, active treatments the patient is undergoing, treatment option taken, outcome of treatment status, and a vector of patient characteristics taken during the observation window 130 are all data points included within the decision point 120.

The decision point 120 relies on the occurrence of events 110 and other healthcare encounters to collect information that was recorded during the provision of treatment. The information pertaining to a patient's characteristics are recorded during a time window through a series of events. The observation window 130 is a time window during which the uncontrolled event 115 is assessed. The status of a patient can be defined at the end of the observation window 130. In some embodiments, the observation window 130 is defined to begin a set number of days before the decision point 120 and end the day before or the day of the decision point 120. The observation window 130 is a flexible window defined relative to the decision point 120. For example, a decision point 120 relating to treatment for type-2 diabetes may correspond to an observation window 130 of twenty one days before the decision point 120. While a decision point 120 relating to treatment for liver disease may correspond to an observation window 130 of fourteen days.

The washout window 140 is a time window along the longitudinal history 100 representing an interval of time where no record of outcomes is recorded. The washout window 140 is used to exclude patient-related information that may be a result of previous treatment options not relating to the treatment options given at the decision point 120. A longer washout window 140 can assure greater internal validity due to more a thorough assessment of the treatment option given at the decision point 120. However, longer washout windows 140 can reduce generalizability and estimate precision. In some embodiments, the washout window 140 is not used to calculate outcome data. For example, some treatment options allow for immediate data extrapolation where the previous treatment option does not affect the results.

The follow-up window 150 is a time window along the longitudinal history 100 representing occurrence of the outcome event 155 is included in the analysis. The time period for the follow-up window 150 can be calculated using treatment specific algorithms, grace periods, exposure extension, and the like. In some embodiments, the follow-up window 150 begins at the time of the decision point 120 or after the washout window 140 time period for which there is no plausible effect of the previous treatment option given.

The outcome event 155 is an illustrative representation of an event 110 for which patient information can be retrieved to determine the effectiveness of the treatment option given at the decision point 120. For example, a treatment option for hypertension was a combination of ACE inhibitors and beta-blockers. The outcome event 155 can represent an event 110 for which a patient's data is relevant in determining whether the ACE inhibitors and beta-blockers are controlling the hypertension.

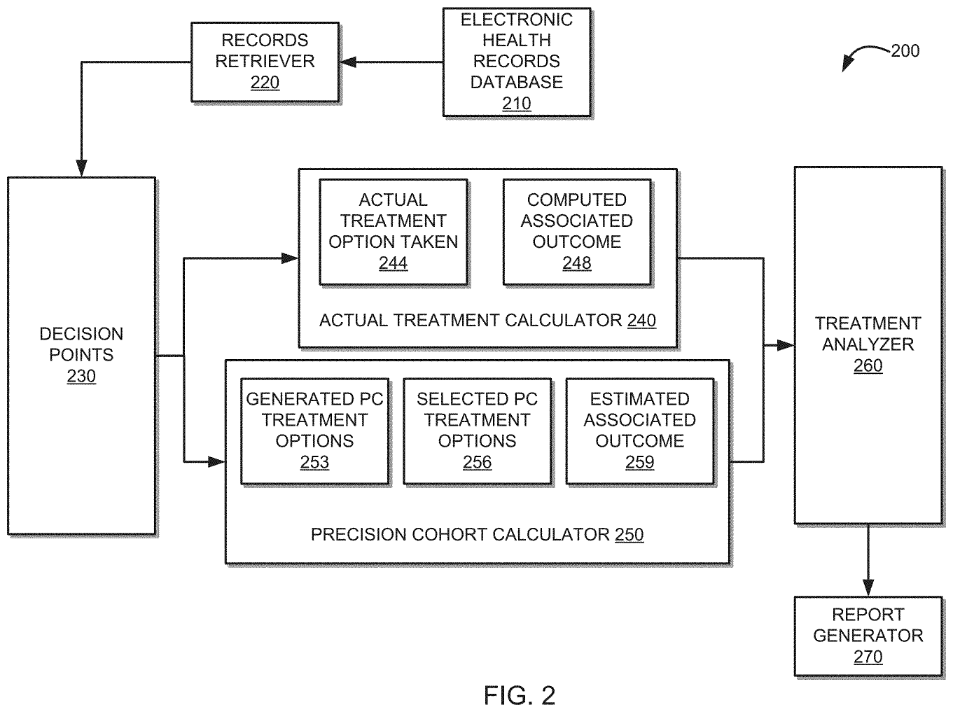

FIG. 2 is a block diagram illustrating a precision cohort analytics system 200 for generating personalized treatment options and associated outcome estimates involving patients, in accordance with embodiments of the present disclosure. The precision cohort analytics system 200 includes electronic health records 210, a records retriever 220, decision points 230 (e.g., which may be the same as, or substantially similar to, decision point 120 of FIG. 1), an actual treatment calculator 240, a precision cohort calculator 250, a treatment analyzer 260, and a report generator 270.

The electronic health records database 210 maintains a collection of patient data including longitudinal history for patients. In some embodiments, the electronic health records database 210 includes information from multiple applications, providers, patients, organizations, and communities. In some embodiments, the electronic health records database 210 spans multiple databases, multiple data store of records, across multiple servers facilitating the storing and retrieval of health records. For example, the electronic health records database 210 can include, but are not limited to, clinic databases, hospital databases, health exchange databases, and health plan records databases. Health records stored within the electronic health records database 210 include, but are not limited to, clinical data and healthcare-related financial data. Clinical data can include any healthcare or medical data particular to a patient, such as lab tests diagnostic tests, clinical encounters, e-visits, and the like.

Clinical data can also include, but is not limited to, health history of a patient, a diagnosis, a clinician assessment, clinician narrative, a treatment, a family history, an immunization record, a medication, age, gender, date of birth, laboratory results, diagnostics, test results, allergies, reactions, procedures performed, history of healthcare visits, healthcare providers, prescriptions, claims data, care process data, and the like. In some embodiments, the electronic health records database 210 includes treatment option outcomes. The outcomes can include medical compliance information referring to the level of compliance of a patient with a prescribed treatment option, such as medications, diet, therapy, follow-up visits, and the like. Healthcare-related financial data can include any financial information pertaining to a patient, such as insurance data, claims data, cost of treatment, payer information, and the like.

The records retriever 220 is a component of the precision cohort analytics system 200 configured to retrieve health records for a population of patients stored within the electronic health records database 210. For example, a patient cohort can be defined for a group of particular patients for which the records retriever 220 can obtain health records relating to the longitudinal histories of the patients within the patient cohort. In some embodiments, the records retriever 220 retrieves health records from multiple electronic health records databases 210 which are stored independently from each other. For example, the records retriever 220 can retrieve clinical data from a clinic locally stored on a server and can also retrieve financial data maintained by a hospital and locally stored on a separate server.

The records retriever 220 is further configured to identify decision points 230 within the information retrieved from the electronic health records database 210. For example, an analysis regarding treatment options for patients with type 2 diabetes is being performed. The records retriever 220 can identify decision points within the records relating treatment options given to the patients with type 2 diabetes.

The actual treatment calculator 240 is a component of the precision cohort analytics system 200 configured to compute actual treatment options taken 244 at the decision points 230 identified by the records retriever 220. By way of example, a patient cohort with coronary heart disease is defined and the records for those patients are retrieved. The decision points 230 from those records can reflect the treatment options taken 244 to control their coronary heart disease. The actual treatment calculator 240 can compute the actual treatment options taken 244 at the decision points 230 identified. These treatment options taken 244 for the coronary heart disease can include treatments such as cholesterol-modifying medications, aspirin medication, beta blockers, calcium channel blockers, ranolazine, nitroglycerin, health changes recommendations, surgery, cardiac rehabilitation, exercise prescriptions, and the like. The dosage, patient characteristics, cost, and other features, can also be included in the actual treatment option taken 244 to assist in determining an overall cost and value of the treatments.

The actual treatment calculator 240 is further configured to compute the associated outcomes 248. The associated outcomes 248 are indications as to the effectiveness of the actual treatment options taken 244 for a condition the patients are attempting to control. The actual treatment calculator can determine an appropriate follow-up window for which patient information and values can attributed to the treatment options taken 244. Once determined, the actual treatment calculator 240 computes the associated outcomes 248 of the actual treatment options taken 244. The outcomes 248 can include patient data, characteristics, symptoms, determination as to whether the condition is under control, and the like.

The precision cohort calculator 250 is a component of the precision cohort analytics system 200 configured to compute precision cohort treatment options 253 for a patient group based on the decision points 230 identified by the records retriever 220. A precision cohort refers to a group of patients who are similar to a given patient group of interest in a clinically meaningful way. Determining whether patients are similar in a clinically meaningful way can be performed in a variety of methods. These methods include, but are not limited to, grouping patients based on a learned similarity metric from patients' records where patients with the same clinical outcome are considered similar, grouping patients based on a learned distance metric to retrieve the K most similar patients and provide a prognosis insight based on the longitudinal history of similar patients, and grouping based on a learned metric from medical professionals feedback.

In some embodiments, the precision cohort calculator 250 applies machine learning models and techniques to determine the precision cohort treatment options 253. For example, a machine learning model can input characteristics of drugs used to treat a particular condition. These characteristics can include, but are not limited to, target proteins, chemical structure, and side effects inputted as features for a drug into the machine learning model. The machine learning model can utilize a machine learning technique to output the precision cohort treatment option 253 for a given precision cohort.

The machine learning model implemented by the precision cohort calculator 250 can be configured to learn from training data (e.g., patient information, clinical data) and generate treatment options and associated outcomes for precision cohorts once trained. The machine learning model is further configured to adjust parameters and weights of features during the training cycle. The machine learning modes can perform predictive analyses, spam detection, pattern detection, image classification, other types of categorical classifications, as well as logistic regressions. The machine learning model can employ different algorithmic methods and techniques to map and label the inputted data. Machine learning algorithms can include, but are not limited to, decision tree learning, association rule learning, artificial neural networks, deep learning, inductive logic programming, support vector machines, clustering, Bayesian networks, reinforcement learning, representation learning, similarity/metric training, sparse dictionary learning, genetic algorithms, rule-based learning, and/or other machine learning techniques.

For example, the machine learning algorithms can implement techniques such as K-nearest neighbor (KNN), learning vector quantization (LVQ), self-organizing map (SOM), logistic regression, ordinary least squares regression (OLSR), linear regression, stepwise regression, multivariate adaptive regression spline (MARS), ridge regression, least absolute shrinkage and selection operator (LASSO), elastic net, least-angle regression (LARS), probabilistic classifier, naïve Bayes classifier, and binary classifier.

In some embodiments, the machine learning model implements deep learning techniques based on artificial neural networks. Deep learning techniques include deep neural networks, deep belief networks, recurrent neural networks, and convolutional neural networks. Deep learning applies techniques which implement multiple layers to progressively extract higher level features from the input.

The machine learning model is further configured to provide a logistic regression type model. This model type can generate a prediction probability for each label predicted. For example, if the machine learning model predicts a treatment option for cardiac rehabilitation, that prediction is accompanied with a prediction probability, or confidence level, the machine learning model has in providing that prediction. The prediction probability can be a percentage ranging from 0 to 100% depending on the confidence of the machine learning model. It should be noted that other forms of prediction probability can also show the confidence level of a predicted label. As the machine learning model is trained, its prediction probabilities can also increase.

In some embodiments, the precision cohort calculator 250 applies machine learning models and techniques to determine a metric for identifying clinically similar patients. For example, supervised similarity learning can input patient feature vectors derived from the information retrieved by the records retriever 220 from the electronic health records database 210. Similar patients determined by the machine learning model can grouped together to comprise the precision cohort while dissimilar patients can be excluded.

The precision cohort calculator 250 is further configured to select precision cohort treatment options 256 for the computed precision cohort and the generated precision cohort treatment options 253. In some embodiments, the precision cohort calculator 250 applies machine learning models and techniques to determine the selected treatment options 256. For example, the machine learning model can incorporate multiple sources of similarity between patients, such as demographics, clinical data, genomic data, and the like as features for the patients. The machine learning model can also incorporate sources of similarity for treatment options relating to the patients. By combining treatment option similarities and patient similarities, an estimated treatment option 256 specifically catered to a particular patient group can be devised.

The precision cohort calculator 250 is further configured to estimate an associated outcome 259 relating to the selected treatment options 256 and the precision cohort. The outcome 259 can be a determination on the effectiveness the selected precision cohort treatment option 256 will have on a patient group. In some embodiments, causal inference is used to determine the associated outcome 259. A causal inference draws a conclusion about a causal connection based on the conditions of the occurrence of an effect. As such, the effect being the selected precision cohort treatment option 256 effectiveness for a given condition. The historical effectiveness of the selected precision cohort treatment option 256 for a given precision cohort can be used as the estimated associated outcome 259.

The treatment analyzer 260 is a component of the precision cohort analytics system 200 configured to analyze the decision points 230 by comparing the actual treatment options 244 with the selected precision cohort treatment options 256 to determine a recommended measure for a patient cohort. The recommended measure can include information relating to the effectiveness the selected precision cohort treatment option 256 has over the actual treatment options 244 taken. The effectiveness can be determined as it relates to outcomes, cost, time, missed opportunities, and potential impact of those missed opportunities. The treatment analyzer 260 is further configured to provide the report generator 270 with the recommended measure calculations.

In some embodiments, the treatment analyzer 260 perform an analysis on a variety of given conditions provided to the treatment analyzer 260. For example, an analysis can be performed on given conditions where all the decision points 230 are analyzed in terms of actual treatment options 244 compared to no treatment change. Another comparison can be performed involving all the decision points 230 with a comparison of the actual associated outcomes 248 and the estimated associated outcomes 259. In some embodiments, the treatment analyzer 260 performs an analysis using decision points where the actual treatment options are not changed and the actual associated outcomes 248 and estimated associated outcomes 259 are compared. The treatment analyzer 260 can be configured to analyze a variety of scenarios depending on the needs of an administrator performing the analysis.

The report generator 270 is a component of the precision cohort analytics system 200 configured to generate precision population analysis reports based on the analysis conducted by the treatment analyzer 260 and the recommended measures generated. In some embodiments, the report generator 270 receives multiple analyses performed by the treatment analyzer 260. For example, the treatment analyzer 260 can perform an analysis involving all the decision point 230 and another analysis where only a select number of decision points 230 are used. Both analyses can be combined or remain separate when the precision population analysis report is generated.

In some embodiments, the report generator 270 emphasizes clinical inertia, missed opportunities for improved outcomes based on treatment option changes, and the potential impact, in terms of time and cost, of those missed opportunities.

It is noted that FIG. 2 is intended to depict the representative major components of an exemplary precision cohort analytics system 200. In some embodiments, however, individual components may have greater or lesser complexity than as represented in FIG. 2, components other than or in addition to those shown in FIG. 2 may be present, and the number, type, and configuration of such components may vary.

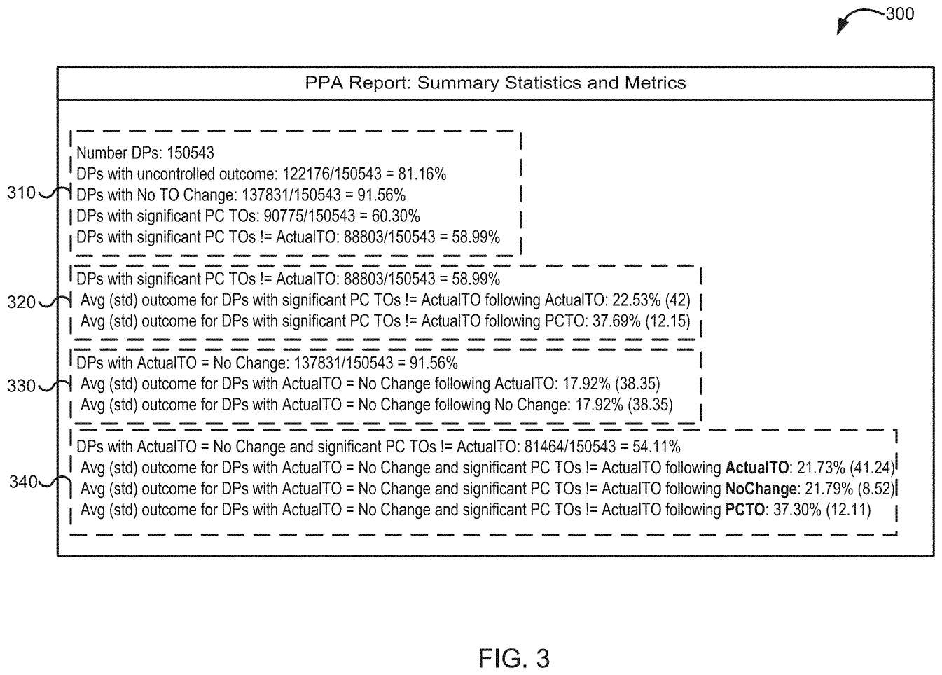

FIG. 3 is an exemplary precision population analysis report illustrating a summary statistic and metrics page 300 for a given population cohort with decision points, in accordance with embodiments of the present disclosure. The summary statistics and metrics page 300 includes information relating to an analysis performed by the treatment analyzer 260 for a given population cohort. The summary statistic and metrics page 300 includes introductory segment 310, a significant change segment 320, a no change segment 330, and a combination segment 340.

The introductory segment 310 includes the number of decision points 230 that were analyzed by the precision cohort analytics system 200. Decision points 230 with an uncontrolled outcome 248 are parsed from the total number of decision points 230 and make up 81.16% of the total number of decision points 230. Decision points 230 with not treatment option change is listed, as well as decision points 230 with precision cohort treatment options indicating a significant different estimated associated outcome 259 to the actual associated outcome 248 are listed.

The significant change segment 320 includes calculations involving the average outcome for decision points with significant precision cohort treatment options that are dissimilar to the actual treatment options provided to the patients. This analysis can represent outcome change based on a patient following the actual treatment option as compared to the precision cohort treatment option.

The no change segment 330 includes calculations to decision points 230 involving no change to their treatment options. The combination segment 340 calculations to decision pointers 230 where there was no change in their actual treatment options, but the precision cohort treatment option was different from the actual treatment option.

It should be understood that the various combinations of statistics and metrics can be displayed on a summary statistics and metrics portion of a precision population analysis report. Depending on the needs of an administrator as they relate to a patient cohort being analyzed, various comparisons can be made between actual treatment options 244, associated outcomes 248, precision cohort treatment options 256, and estimated associated outcomes 259.

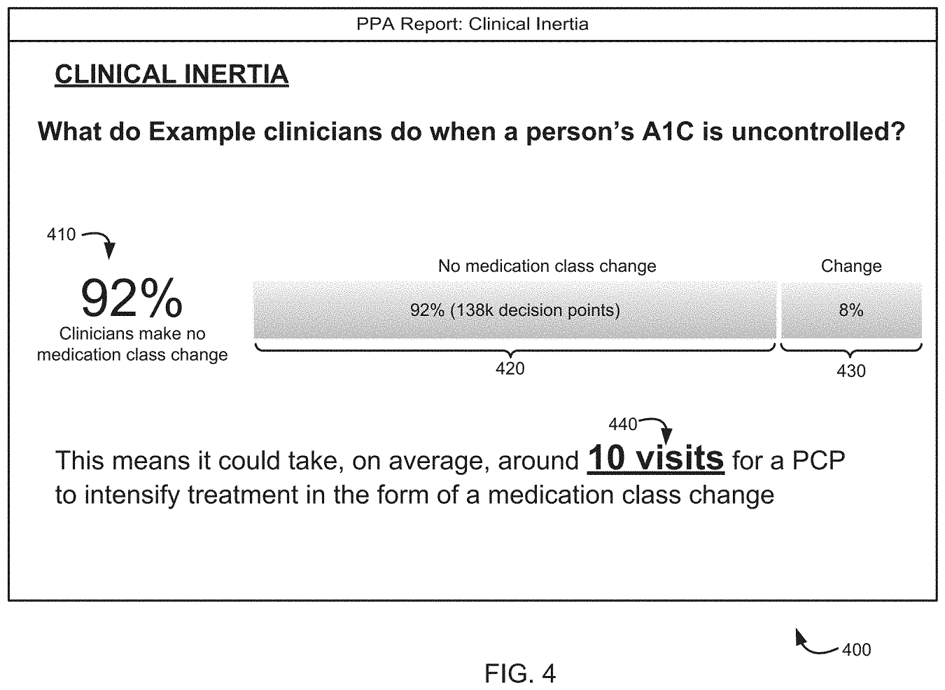

FIG. 4 is an exemplary precision population analysis report illustrating a summary clinical inertia page 400 for a given population cohort with decision points, in accordance with embodiments of the present disclosure. The clinical inertia page 400 includes a percentage indicator 410, no change indicator bar 420, a change bar 430, and an impact statement 440.

The percentage indicator 410 represents the percentage of clinicians that made no treatment option changes when a patient's A1C remained uncontrolled. The percentage indicator 410 is a numerical indication of an analysis performed by the treatment analyzer 260 for a patient cohort consisting of 138,000 decision points. The no change indicator bar 420 additionally reflects the lack of change by clinicians in situations where a patient's A1C is uncontrolled. Only 8% of clinicians altered their treatment options as represented by the change bar 430. To further emphasis the clinical inertia identified in this exemplary patient cohort, the impact statement 440 displays an additional estimation that on average, 10 visits to a primary care physician are required by a patient before a different treatment option is selected.

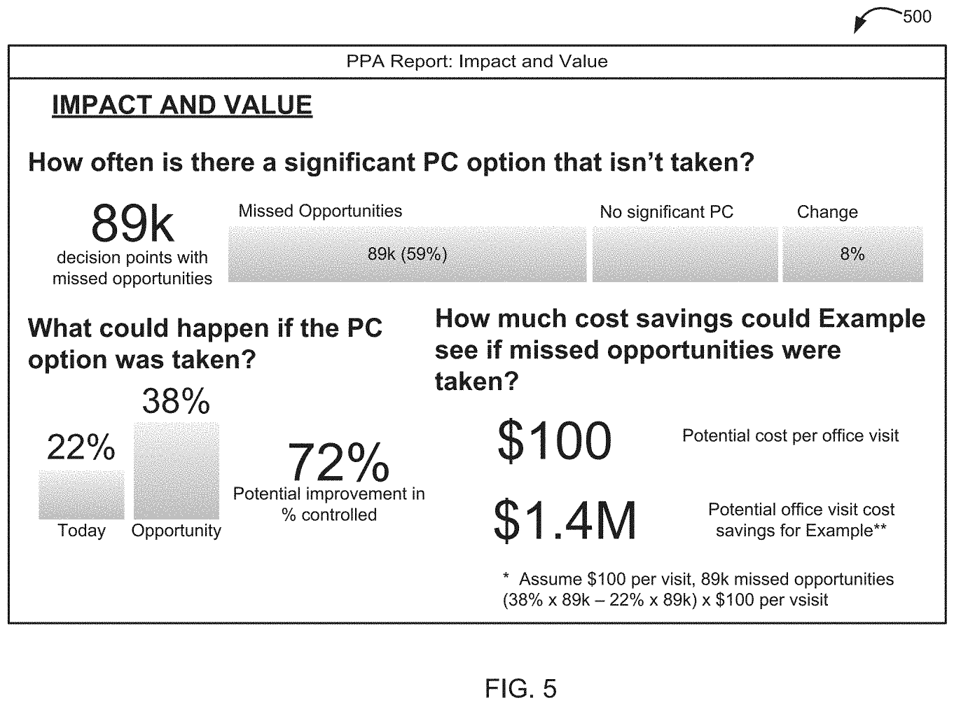

FIG. 5 is an exemplary precision population analysis report illustrating an impact and value page 500 for a given population cohort with decision points, in accordance with embodiments of the present disclosure.

The impact and value page 500 illustrates various segments where a significant precision cohort treatment option can impact a patient's condition and/or reduce costs associated with a treatment option. Taken from a patient cohort, 89,000 missed opportunities were determined. That 89,000 missed opportunities for an exemplary condition translated to 72% improvement of condition for patients and a savings of $1.4 million dollars to an exemplary health care provider.

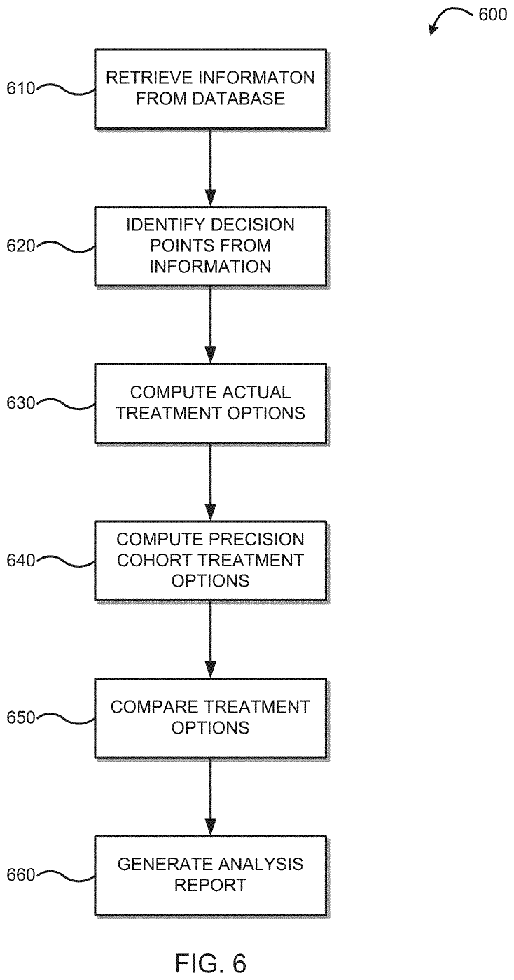

FIG. 6 is a flow diagram illustrating a process 600 for generating personalized treatment options and associated outcome estimates involving patients, in accordance with embodiments of the present disclosure. The process 600 may be performed by hardware, firmware, software executing on at least one processor, or any combination thereof. For example, any or all of the steps of the process 600 may be performed by one or more computing devices.

The process 600 begins by the records retriever 220 retrieving information from an electronic health records database 210 relating to a patient cohort. This is illustrated at step 610. The information retrieved can relate to the health records relating to the longitudinal histories of the patients within the patient cohort. In some embodiments, the records retriever 220 retrieves information relating to a patient cohort from multiple sources. The source can include servers, databases, and other records stored independently from each other. For example, the records retriever 220 can retrieve pharmaceutical data from a healthcare provider's database and clinical data from a clinical database.

The records retriever 220 identifies decision points 230 relating to a condition, illness, symptom, or ailment, the patients exhibit that is reflected in the information retrieved from the electronic health records database 210. This is illustrated at step 620. The decision point 230 can include information such as the date of the event, active treatments at the time of the decision point 230, actual treatment options taken 244 as a result of the decision point 230, associated outcomes 248, and various patient characteristics relevant to the condition for which the decision point 230 relate to.

In some embodiments, the records retriever 220 additionally retrieves characteristic features for each patient in the patient cohort relating to the clinical treatment decision points. The characteristic features can include features of the patient relating to a condition pertaining to the decision point 230. For example, the decision points may be identified for patients with congestive heart failure. The characteristic features that are then retrieved can relate to pertinent characteristics such as age, gender, weight, medication, and the like of the patient.

In some embodiments, the records retriever 220 additionally retrieves outcome features associated with outcomes relating to the clinical treatment decision points. The outcome features can include features such as the treatment options given, the result of the treatment options, the time taken for the treatment to be effective, and the like.

In some embodiments, the characteristic features and the outcome features retrieved by the records retriever 220 are provided to a machine learning model implemented by the precision cohort calculator 250 for determining precision cohort treatment options 253 and associated outcomes 259.

The actual treatment calculator 240 computes the actual treatment options from the decision points. This is illustrated at step 630. The decision points 230 reflect some interaction with a medical provider that prescribes a treatment for a given condition. The medical provider can choose to continue with a current treatment option, change the treatment option, or completely cancel a treatment option. The actual treatment calculator 240 can analyze the information at the time of the decision point 230 to determine the actual treatment option given by the medical provider. In some embodiments, the decision points 230 include the actual treatment options taken 244.

The actual treatment calculator 240 computes the actual treatment options taken for each of the decision points 230 identified by the records retriever 220. This is illustrated at step 630 In some embodiments, the actual treatment options taken are included in the decision point 230. In some embodiments, the actual treatment calculator 240 searches the information to determine the treatment option a medical professional selected at the time of the decision point 230. For example, a decision point 230 may refer to a clinical visit for patients with congestive heart failure that is uncontrolled. The course of action the medical professional treats the patient with can be determined as the actual treatment option taken 244.

In some embodiments, the actual treatment calculator 240 additionally computes the associated outcomes 248 of the actual treatment options taken 244 for each of the decision points 230. The associated outcomes 248 can be the condition of the patient as a result of the actual treatment option taken 244. The actual treatment calculator 240 can determine a follow-up window for a given condition and treatment option that can provide an accurate outcome for that treatment. Based on that time window, a patient's characteristics can be analyzed to determine the associated outcome 248.

The precision cohort calculator 250 computes the precision cohort treatment options relating to the decision points 230 using precision cohort analytics on a patient group. This is illustrated at step 640. The patient groups can be a population cohort, a different patient cohort, or an individual patient. In some embodiments, the precision cohort calculator 250 utilizes machine learning models and techniques when applying precision cohort analytics to the patient group. For example, the precision cohort calculator 250 can apply a machine learning model to generate a patient group based on their similarities as it pertains to their health and condition relating to the decision points 230 identified.

In some embodiments, the precision cohort calculator 250 applies precision cohort analytics to each of the decision points 230 to generate personalized treatment during step 640. In some embodiments, the precision cohort calculator 250 implements a machine learning model to analyze the actual treatment option taken 244 and the condition of the patient at the time of the decision point 230 and use that information as features into the machine learning model to generate the personalized treatment option as an output. This can be repeated for each decision point 230.

In some embodiments, the precision cohort calculator 250 selects treatment option suggestions taken from the personalized treatment options. The selections can be based on whether the personalized treatment option has a significant impact on the condition of the patient, the cost to the healthcare provider or patient, or both the condition and cost. In some embodiments, the precision cohort calculator 250 utilizes a machine learning model to determine the treatment option suggestions. The machine learning model can input the personalized treatment options and a patient group as inputs to generate treatment option suggestion which factor in both treatment options and patient group when making a determination on the treatment option suggestions.

In some embodiments, the precision cohort calculator 250 estimates the associated outcomes for the treatment option suggestions. This can be performed through historical analysis of the treatment option suggestions actual use cases for comparable patients. For example, if a treatment option suggestion selected is alendronate for a patient with osteoporosis. An analysis can be performed for other patients with similar characteristics and conditions as the patient being treated with alendronate and see their outcomes to estimate the associated outcome for the treatment option suggestion. In some embodiments, a causal inference is used to estimate the associated outcomes.

The treatment analyzer 260 compares the actual treatment options and the precision treatment options for each of the decision points 230 to determine recommended measures that can be taken by patients and healthcare providers. This is illustrated at step 650. In some embodiments, the comparison is made to determine clinical inertia. For a given condition determined at a decision point 230, an analysis can be performed to determine how often the condition remains uncontrolled within the patient. At these particular decision points 230, a further analysis can be performed to determine when a medical professional choose to not change the treatment for the patients with the uncontrolled condition. At the decision points 230 where a condition remains uncontrolled and a medical professional chooses to not change treatment can be indicative of clinical inertia.

In some embodiments, the comparison made by the treatment analyzer 260 is used to determine whether there are missed opportunities for improved outcomes within the patient cohort. An analysis can be made on the decision points 230 where no treatment change was determined and compare those patients with the selected precision cohort treatment options 256 to see if the estimated associated outcomes 259 provide for a more positive outcome for the patients. Further, a determination can be made as to how often the selected precision cohort treatment options 256 differ from the actual treatment options 244 including those treatment options that are not changed at the decision points 230. The decision points 230 where the estimated associated outcomes 259 for the corresponding selected precision cohort treatment options 256 show an improvement over the associated outcomes 248 can be indicative of missed opportunities for improvement.

In some embodiments, a potential impact, relating to time and cost, comparison can be made to the decision points 230 determined to be missed opportunities for improvement. The treatment analyzer 260 can compare the estimated associated outcomes 259 to the computed associated outcomes 248 for those decision points 230 to determine a difference in outcome. Based on that difference, a determination can be made as to how much additional time, in terms of patient years, does the condition remain uncontrolled as a result of the missed opportunities. Furthermore, a determination can be made as to the financial impact that has to both the patient and the healthcare provider.

In some embodiments, the clinical inertia comparison, the missed opportunities comparison, and the potential comparison analysis can be performed by repeating the analysis on a different patient group.

A precision population analysis report is generated by the report generator 270. This is illustrated at step 660. The analysis report can contain the comparisons and information compiled by the treatment analyzer 260. For example, the precision population analysis report may include information as described in FIGS. 3-5. In some embodiments, the report generator 270 includes embodiment comparisons performed by the treatment analyzer. For example, the precision population analysis report can include a clinical inertia comparison and a potential impact comparison. In some embodiments, the report generator 270 generates a precision population analysis report based on a predetermined selection of comparisons. In some embodiments, the precision population analysis report is provided to an individual or corporation. For example, the precision population analysis report can be provided to a patient, a medical professional, a health care provider, or a research facility.

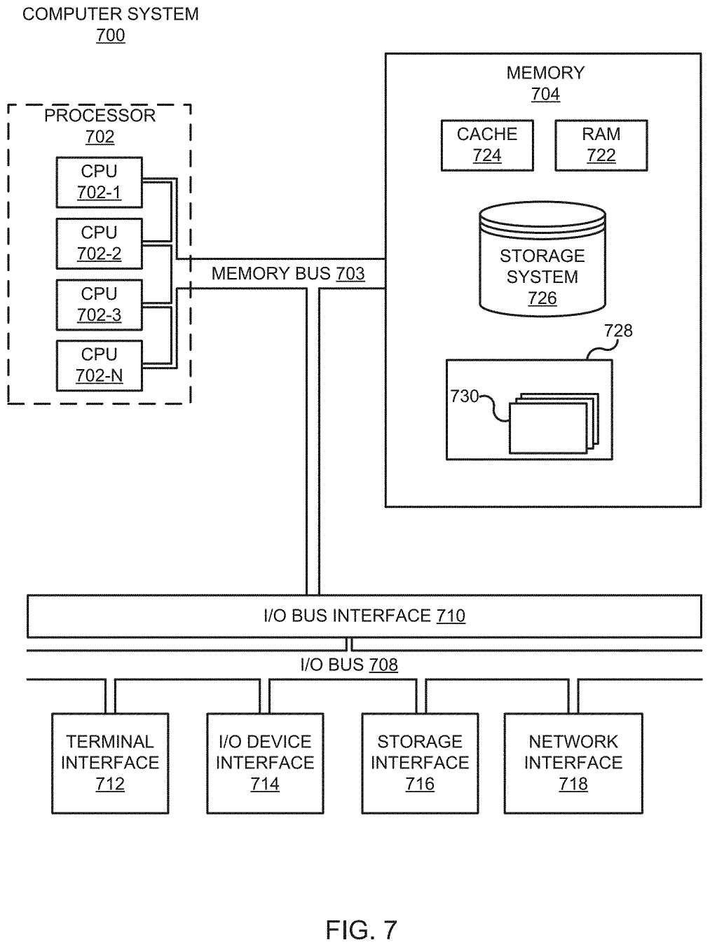

Referring now to FIG. 7, shown is a high-level block diagram of an example computer system 700 (e.g., precision cohort analytics system 200) that may be used in implementing one or more of the methods, tools, and modules, and any related functions, described herein (e.g., using one or more processor circuits or computer processors of the computer), in accordance with embodiments of the present disclosure. In some embodiments, the major components of the computer system 700 may comprise one or more processors 702, a memory 704, a terminal interface 712, a I/O (Input/Output) device interface 714, a storage interface 716, and a network interface 718, all of which may be communicatively coupled, directly or indirectly, for inter-component communication via a memory bus 703, a I/O bus 708, and an I/O bus interface 710.

The computer system 700 may contain one or more general-purpose programmable central processing units (CPUs) 702-1, 702-2, 702-3, and 702-N, herein generically referred to as the processor 702. In some embodiments, the computer system 700 may contain multiple processors typical of a relatively large system; however, in other embodiments the computer system 700 may alternatively be a single CPU system. Each processor 701 may execute instructions stored in the memory 704 and may include one or more levels of on-board cache.

The memory 704 may include computer system readable media in the form of volatile memory, such as random-access memory (RAM) 722 or cache memory 724. Computer system 700 may further include other removable/non-removable, volatile/non-volatile computer system storage media. By way of example only, storage system 726 can be provided for reading from and writing to a non-removable, non-volatile magnetic media, such as a “hard drive.” Although not shown, a magnetic disk drive for reading from and writing to a removable, non-volatile magnetic disk (e.g., a “floppy disk”), or an optical disk drive for reading from or writing to a removable, non-volatile optical disc such as a CD-ROM, DVD-ROM or other optical media can be provided. In addition, the memory 704 can include flash memory, e.g., a flash memory stick drive or a flash drive. Memory devices can be connected to memory bus 703 by one or more data media interfaces. The memory 704 may include at least one program product having a set (e.g., at least one) of program modules that are configured to carry out the functions of various embodiments.

Although the memory bus 703 is shown in FIG. 7 as a single bus structure providing a direct communication path among the processors 702, the memory 704, and the I/O bus interface 710, the memory bus 703 may, in some embodiments, include multiple different buses or communication paths, which may be arranged in any of various forms, such as point-to-point links in hierarchical, star or web configurations, multiple hierarchical buses, parallel and redundant paths, or any other appropriate type of configuration. Furthermore, while the I/O bus interface 710 and the I/O bus 708 are shown as single respective units, the computer system 700 may, in some embodiments, contain multiple I/O bus interface units, multiple I/O buses, or both. Further, while multiple I/O interface units are shown, which separate the I/O bus 708 from various communications paths running to the various I/O devices, in other embodiments some or all of the I/O devices may be connected directly to one or more system I/O buses.

In some embodiments, the computer system 700 may be a multi-user mainframe computer system, a single-user system, or a server computer or similar device that has little or no direct user interface but receives requests from other computer systems (clients). Further, in some embodiments, the computer system 700 may be implemented as a desktop computer, portable computer, laptop or notebook computer, tablet computer, pocket computer, telephone, smart phone, network switches or routers, or any other appropriate type of electronic device.

It is noted that FIG. 7 is intended to depict the representative major components of an exemplary computer system 700. In some embodiments, however, individual components may have greater or lesser complexity than as represented in FIG. 7, components other than or in addition to those shown in FIG. 7 may be present, and the number, type, and configuration of such components may vary.

One or more programs/utilities 728, each having at least one set of program modules 730 may be stored in memory 704. The programs/utilities 728 may include a hypervisor (also referred to as a virtual machine monitor), one or more operating systems, one or more application programs, other program modules, and program data. Each of the operating systems, one or more application programs, other program modules, and program data or some combination thereof, may include an implementation of a networking environment. Programs 728 and/or program modules 730 generally perform the functions or methodologies of various embodiments.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

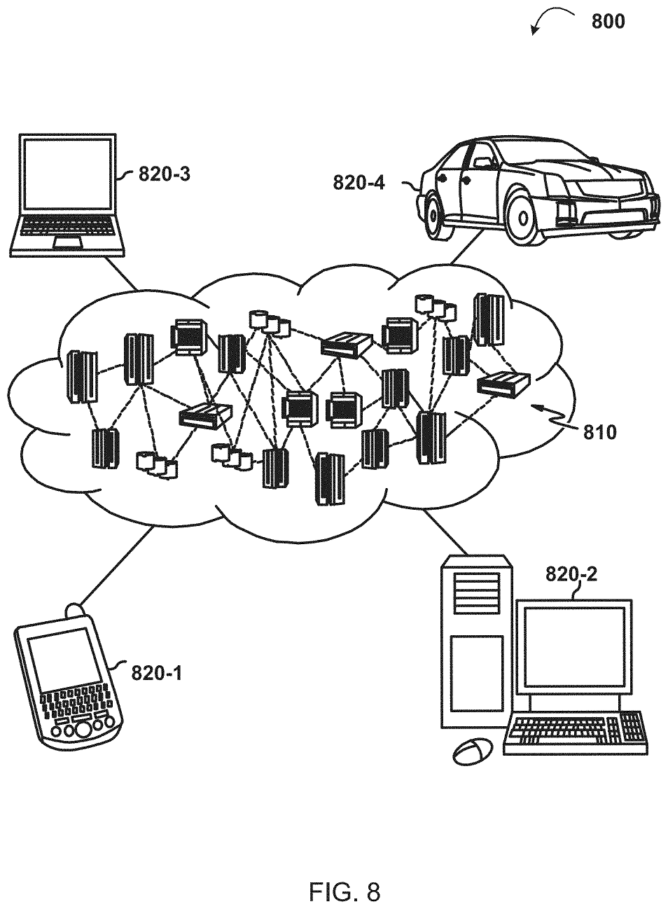

Referring now to FIG. 8, illustrative cloud computing environment 800 is depicted. As shown, cloud computing environment 800 includes one or more cloud computing nodes 810 with which local computing devices used by cloud consumers, such as, for example, personal digital assistant (PDA) or cellular telephone 820-1, desktop computer 820-2, laptop computer 820-3, and/or automobile computer system 820-4 may communicate. Nodes 810 may communicate with one another. They may be grouped (not shown) physically or virtually, in one or more networks, such as Private, Community, Public, or Hybrid clouds as described hereinabove, or a combination thereof. This allows cloud computing environment 800 to offer infrastructure, platforms and/or software as services for which a cloud consumer does not need to maintain resources on a local computing device. It is understood that the types of computing devices 820-1 to 820-4 shown in FIG. 8 are intended to be illustrative only and that computing nodes 810 and cloud computing environment 800 can communicate with any type of computerized device over any type of network and/or network addressable connection (e.g., using a web browser).

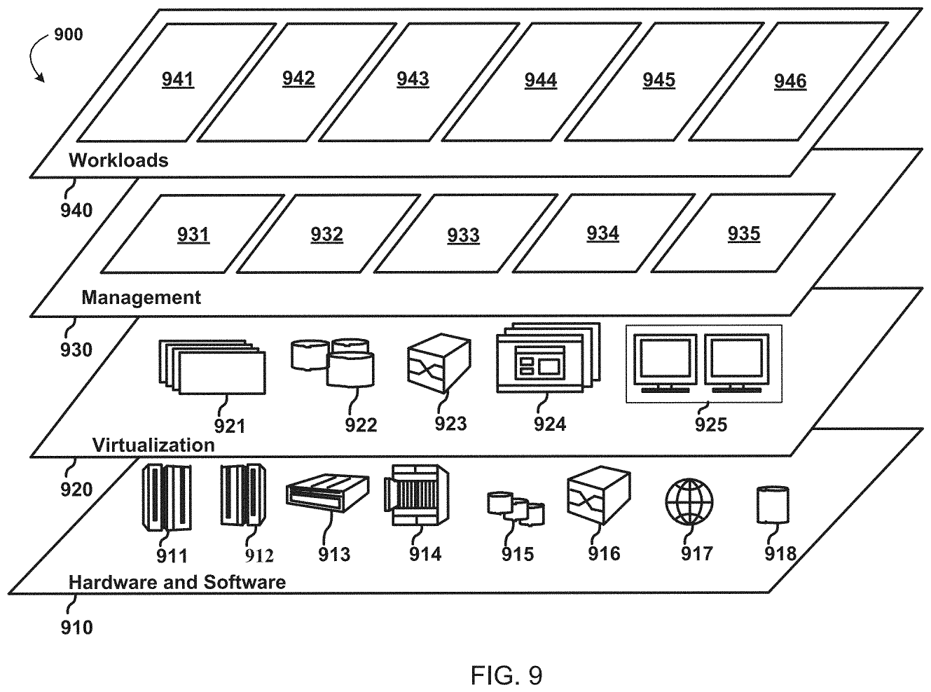

Referring now to FIG. 9, a set of functional abstraction layers 900 provided by cloud computing environment 800 (FIG. 8) is shown. It should be understood in advance that the components, layers, and functions shown in FIG. 9 are intended to be illustrative only and embodiments of the invention are not limited thereto. As depicted, the following layers and corresponding functions are provided:

Hardware and software layer 910 includes hardware and software components. Examples of hardware components include: mainframes 911; RISC (Reduced Instruction Set Computer) architecture-based servers 912; servers 913; blade servers 914; storage devices 915; and networks and networking components 916. In some embodiments, software components include network application server software 917 and database software 918.

Virtualization layer 920 provides an abstraction layer from which the following examples of virtual entities may be provided: virtual servers 921; virtual storage 922; virtual networks 923, including virtual private networks; virtual applications and operating systems 924; and virtual clients 925.

In one example, management layer 930 may provide the functions described below. Resource provisioning 931 provides dynamic procurement of computing resources and other resources that are utilized to perform tasks within the cloud computing environment. Metering and Pricing 932 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 933 provides access to the cloud computing environment for consumers and system administrators. Service level management 934 provides cloud computing resource allocation and management such that required service levels are met. Service Level Agreement (SLA) planning and fulfillment 935 provide pre-arrangement for, and procurement of, cloud computing resources for which a future requirement is anticipated in accordance with an SLA.

Workloads layer 940 provides examples of functionality for which the cloud computing environment may be utilized. Examples of workloads and functions which may be provided from this layer include: mapping and navigation 941; software development and lifecycle management 942; virtual classroom education delivery 943; data analytics processing 944; transaction processing 945; and precision cohort analytics 946 (e.g., the precision cohort analytics system 200).

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A computer-implemented method comprising:
retrieving information from an electronic health records database relating to a patient cohort;
identifying decision points from the information retrieved;
computing actual treatment options from the decision points;
computing precision cohort treatment options relating to the decision points using precision cohort analytics on a patient group;
analyzing the decision points by comparing the actual treatment options with the precision cohort treatment options for each of the decision points to determine recommended measures; and
generating a precision population analysis report based on the recommended measures.

2. The computer-implemented method of claim 1, wherein retrieving the information the electronic health records database comprises:
identifying clinical treatment decision points in the information retrieved from the electronic health records database, wherein the clinical treatment decision points are listed as events on longitudinal histories relating to patients;
retrieving characteristic features for each patient in the patient cohort relating to the clinical treatment decision points; and
retrieving outcome features associated with outcomes relating to the clinical treatment decision points.

3. The computer-implemented method of claim 1, wherein computing the actual treatment options comprises:
identifying actual treatment options taken for each of the decision points; and
computing associated outcomes for each of the decision points based on the actual treatment options taken.

4. The computer-implemented method of claim 1, wherein computing the precision cohort treatment options comprises:
applying precision cohort analytics to each of the decision points to generate personalized treatment options;
selecting treatment option suggestions from the personalized treatment options; and
estimating associated outcomes for the treatment option suggestions.

5. The computer-implemented method of claim 4, wherein estimating the associated outcomes is based on a causal inference.

6. The computer-implemented method of claim 1, wherein the patient group is chosen from the group consisting of a population cohort, a different patient cohort, a patient panel, and from an individual patient.

7. The computer-implemented method of claim 1, wherein analyzing the decision points by comparing the actual treatment options with the precision cohort treatment options comprises:
determining a clinical inertia by analyzing the decision points for no treatment change;
comparing the actual treatment options with the precision cohort treatment options to quantify an amount of treatment decision overlap;
comparing actual treatment outcomes with precision cohort outcomes to quantify a difference in expected control;
estimating a potential impact relating to the difference in expected control; and
repeating the analysis on a different patient group.

8. A computer program product comprising a computer readable medium having program instructions embodied therewith, the program instructions executable by a processor to cause the processor to perform a method comprising:
retrieving information from an electronic health records database relating to a patient cohort;
identifying decision points from the information retrieved;
computing actual treatment options from the decision points;
computing precision cohort treatment options relating to the decision points using precision cohort analytics on a patient group;
analyzing the decision points by comparing the actual treatment options with the precision cohort treatment options for each of the decision points to determine recommended measures; and
generating a precision population analysis report based on the recommended measures.

9. The computer program product of claim 8, wherein retrieving the information the electronic health records database comprises:
identifying clinical treatment decision points in the information retrieved from the electronic health records database, wherein the clinical treatment decision points are listed as events on longitudinal histories relating to patients;
retrieving characteristic features for each patient in the patient cohort relating to the clinical treatment decision points; and
retrieving outcome features associated with outcomes relating to the clinical treatment decision points.

10. The computer program product of claim 8, wherein computing the actual treatment options comprises:
identifying actual treatment options taken for each of the decision points; and
computing associated outcomes for each of the decision points based on the actual treatment options taken.

11. The computer program product of claim 8, wherein computing the precision cohort treatment options comprises:
applying precision cohort analytics to each of the decision points to generate personalized treatment options;
selecting treatment option suggestions from the personalized treatment options; and
estimating associated outcomes for the treatment option suggestions.

12. The computer program product of claim 11, wherein estimating the associated outcomes is based on a causal inference.

13. The computer program product of claim 8, wherein the patient group is chosen from the group consisting of a population cohort, a separate patient cohort, a patient panel, and from an individual.

14. The computer program product of claim 8, wherein analyzing the decision points by comparing the actual treatment options with the precision cohort treatment options comprises:
determining a clinical inertia by analyzing the decision points for no treatment change;
comparing the actual treatment options with the precision cohort treatment options to quantify an amount of treatment decision overlap;
comparing actual treatment outcomes with precision cohort outcomes to quantify a difference in expected control;
estimating a potential impact relating to the difference in expected control; and
repeating the analysis on a different patient group.

15. A precision cohort analytics system comprising:
at least one processor;
at least one memory component;
a records retriever configured to retrieve information from an electronics health records database relating to a patient cohort;
a treatment calculator configured to compute actual treatment options from decision points identified in the information on a patient group;
a precision cohort calculator configured to compute precision cohort treatment options from the decision points;
a treatment analyzer configured to analyze the decision points by comparing the actual treatment options with the precision cohort treatment options to determine a recommended measure for the patient cohort; and
a report generator configured to generate a precision population analysis report based on the recommended measures determined.

16. The precision cohort analytics system of claim 15, wherein the records retriever is further configured to identify clinical treatment decision points in the information retrieved from the electronic health records database, wherein the clinical treatment decision points are listed as events on longitudinal histories relating to patients.

17. The precision cohort analytics system of claim 15, wherein the treatment calculator is further configured to identify actual treatment options taken for each of the decision points and compute an associated outcome for each of the decision points based on the actual treatment options taken.

18. The precision cohort analytics system of claim 15, where the precision cohort calculator is further configured to apply precision cohort analytics to each of the decision points to generate personalized treatment options.

19. The precision cohort analytics system of claim 15, wherein the patient group is chosen from the group consisting of a population cohort, a separate patient cohort, a patient panel, and from an individual.

20. The precision cohort analytics system of claim 15, wherein the treatment analyzer is further configured to determine a clinical inertia and to estimate a potential impact relating to the actual treatment options and the precision cohort treatment options.
